(19) United States
(12) Patent Application Publication
(10) Pub. No.: US 2020/0020432 A1
(43) Pub. Date: Jan. 16, 2020

(54) PATIENT INFORMATION SYSTEM

(71) Applicant: Dalin Tzu Chi Hospital, Buddhist Tzu Chi Medical Foundation, Chiayi County (TW)

(72) Inventor: PO-LIANG CHENG, Chiayi County (TW)

(21) Appl. No.: 16/034,792

(22) Filed: Jul. 13, 2018

(51) Int. Cl. G16H 10/65; G16H 80/00

(52) U.S. Cl. CPC G16H 10/65; G16H 80/00

(57) ABSTRACT

A patient information system includes a patient information sharing platform, a terminal device, and an identification label. The patient information sharing platform is configured for storing a patient data and a patient status data. When a communication sensing module of the terminal device is connected with the identification label through a sensing connection, the terminal device is configured for acquiring or editing the patient data and the patient status data. Therefore, the patient related information is efficiently acquired by the user.

### BACKGROUND OF THE INVENTION

### 1. Field of the Invention

The present invention relates to information systems, and more particularly, to a patient information system.

### 2. Description of the Related Art

In a large scale accident, medical staff from various hospitals arrive the scene of the accident for rescuing a large amount of patients. A medical personnel has to efficiently identify the identity of each patient, so as to provide different treatment procedures according to the different injury levels of the patients.

Regarding a conventional procedure for handling the large amount of patients, the medical staff usually identify the patient identities and carry out the medical tag registration manually, and provide treatments according to different medical examination levels. However, manually identifying the patient identities not only prolongs the rescue duration, but also easily cause mistaken identification and registrations. As a result, medical malpractice might occur due to the misidentification of patients.

Also, patient information acquired by manual medical examination and medical tag registration are unable to be shared for all medical staff. Therefore, a single patient might undergo the same identification process by different medical personnel, delaying the rescuing procedure and causing a waste of resource.

Further, before a large amount of patients are delivered to the hospital, the medical staff in the hospital have to be generally informed about the patient amount and the scale of the accident according to the information from the medical personnel at the scene of the accident. However, the medical staff in the hospital are unable to know the injury detail of each patient. Thus, when a large amount of patients, the medical staff in the hospital have to confirm the patient identity and carry out the medical examination again. Also, the medical staff in the hospital are unable to know if the patient has received an initial treatment at the scene of the accident. As a result, the medical staff in the hospital face a chaotic situation, and the medical treatment efficiency is lowered.

### SUMMARY OF THE INVENTION

For improving the issues above, a patient information system is disclosed by the present invention. By use of the sensing connection between the terminal device and the identification label, the patient information is timely updated, facilitating a highly efficient treatment process.

A patient information system in accordance with an embodiment of the present invention comprises:

a patient information sharing platform set up on a server, the patient data sharing platform storing a patient data and a patient status data;

a terminal device connected with the patient information sharing platform through the internet, the terminal device comprising a communication sensing module; and

an identification label providing a sensing identification function and an data transaction function, wherein when the identification label is sensed to be connected with the communication sensing module, the terminal device is able to acquire and edit the patient data and the patient status data.

With such configuration, when an accident occurs, the medical staff are allowed to identify the patient identities at the scene of the accident, and then establish the patient data and patient status data in the patient information sharing platform. Afterward, the medical staff in the subsequent state are able to efficiently identify and confirm the treatment status of the patients through the terminal device, achieving a highly efficient treatment process.

In an embodiment of the present invention, the patient information sharing platform includes a location module, and the terminal device includes a positioning module. The positioning module acquires a current location signal of the terminal device, and the terminal device transmits the current location signal which represents the current location of the patient to the location module, wherein the location module transmits the current location module to the patient stats information. Therefore, the commander at the scene of the accident is able to known the locations of each medical personnel and each patient according to the current location signal displayed on the location module, thereby determining a support needed location, facilitating an efficient rescue.

In an embodiment of the present invention, the patient information sharing platform comprises a hospital data module, and the hospital data module has a plurality of treatment end information. The patient status data has one of the corresponding treatment end information. Therefore, the commander at the scene of the accident is allowed to arrange each patient to receive treatment at a suitable hospital based the treatment end information, the amount of patients, and the respective injury levels. Further, family members of the patients are able to know the hospital accepting the target patient according to the patient status data.

### BRIEF DESCRIPTION OF THE DRAWINGS

FIG. 1 is a block diagram illustrating the connection of the patient information system in accordance with an embodiment of the present invention.

FIG. 2 is a schematic view illustrating the system structure of the patient information system in accordance with an embodiment of the present invention.

FIG. 3 is a schematic view illustrating the user applying the terminal device to sense the identification label.

### DETAILED DESCRIPTION OF THE INVENTION

The aforementioned and further advantages and features of the present invention will be understood by reference to the description of the preferred embodiment in conjunction with the accompanying drawings where the components are illustrated based on a proportion for explanation but not subject to the actual component proportion.

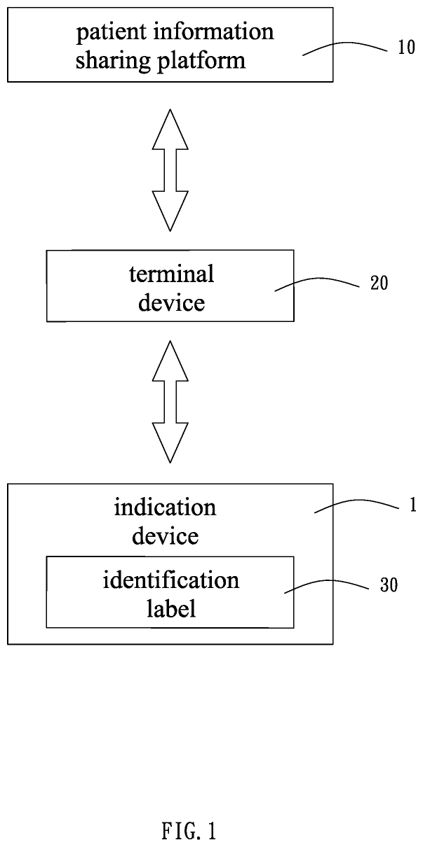

Referring to FIG. 1 to FIG. 3, a patient information system in accordance with an embodiment of the present invention comprises a patient information sharing platform 10, a terminal device 20, and an identification label 30. The patient information sharing platform 10 is set up on a server, and providing access to at least one user 3 through the internet, such that the user 3 is able to access the patient information sharing platform 10 by use of the terminal device 20. The terminal device 20 is a portable device, such as a smart phone or a table computer.

Also, the patient information system further includes an identification label 30, which provides a sensing identification function and a data transaction function. The identification label 30 is disposed on an indication device 1, which is worn on the patient 2. In an embodiment of the present invention, the indication device 1 is a wristband, and the identification label 30 is an NFC (near field communication) label.

In addition, the terminal device 20 includes a communication sensing module 21. When the communication sensing module 21 is connected with the identification label 30 on the indication device 1 by use of the near field communication. The terminal device 20 is allowed to acquire or edit the patient data 11 and the patient status data 12 stored on the patient information sharing platform 10. In the embodiment of the present invention, the user 3 wears the indication device 1 on the patient 2, wherein the user 3 is allowed to be a medical personnel, the rescue commander, one of a medical staff in the hospital, a medical personnel on the ambulance, or a non-medical personnel (such as a family member of the patient 2).

The patient information sharing platform 10 comprises a patient data module 13, a location module 14, a hospital data module 15, an account module 16, and an authorization module 17.

The patient data module 13 stores the patient data 11 and the patient status data 12. The patient data 11 includes an identification data, a treatment record data, and a pattern corresponding to the identification label 30. In an embodiment of the present invention, when the user 3 who is a medical personnel wears the indication device 1 on the patient 2, the user 3 senses the identification label 30 through the terminal device 20 with a sensing connection, such that the user 3 is allowed to establish the patient data 11 and patient status data 12 in the patient data module 13 through the terminal device 20. Also, the user 3 saves the identification label 30 corresponding to the indication device 1 in the patient data 11. The patient data 11 is allowed to be chosen from a text, image, video, or combination thereof. Also, the patient data 11 is allowed to include identification related data, such as the name, gender, and age of the patient 2. The patient status data 12 is allowed to be chosen from a text, image, video, or combination thereof.

The location module 14 is configured to display the location related information. In an embodiment of the present invention, the terminal device 20 includes a positioning module 22 for acquiring a current location signal of the terminal device 20. The positioning module 22 refers to a GPS module. The terminal device 20 transmits the current location signal to the location module 14, such that the location module 14 displays the current location signal of the terminal device 20. Also, the location module 14 is able to transmit the current location signal to the patient status data 12.

The hospital data module 15 includes a plurality of treatment end information 151, wherein the treatment end information 151 includes hospital related information, such as the name and classification of the hospital. In an embodiment of the present invention, the user 3 is able to arrange the patient 2 to be delivered to a suitable hospital according to the injury status of the patient 2, and the user 3 is also able to use the terminal device 20 to establish the treatment end information 151 arranged for the patient 2 in the patient status data 12.

The account module 16 allows the user to register a user information 161 with the account module 16 through the terminal device 20.

The authorization module 17, according to the user information 161 in the account module 16, limits the authority of the user 3 viewing the data on the patient information sharing platform 10. More specifically, when the user 3 is a non-medical personnel, the authorization module 17 imposes the limitation upon the viewable data content for the user 3. For example, the user 3 is thus only allowed to view the patient status data 12 or the identification data in the patient data 11. Further, when the user 3 is a medical personnel, the user 3 is allowed to view all related data of the patient 2, including the patient status data 12, patient data 11, and the location displayed by the location module 14.

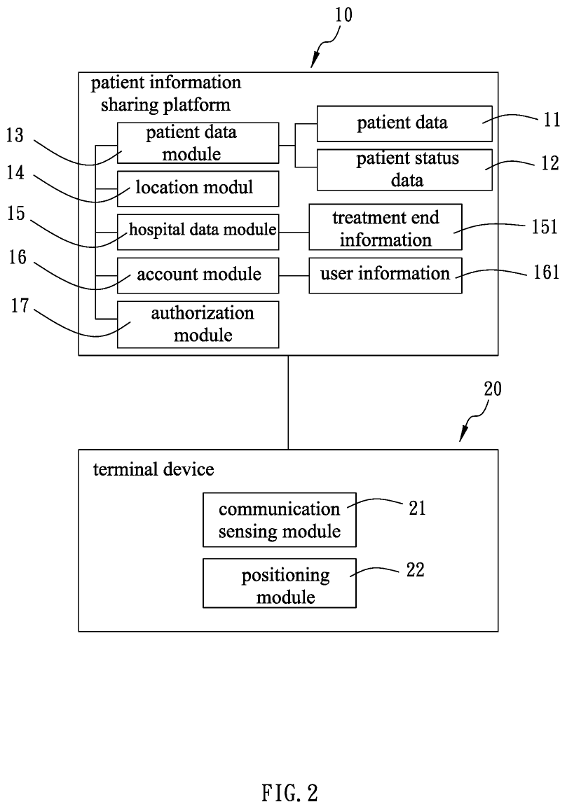

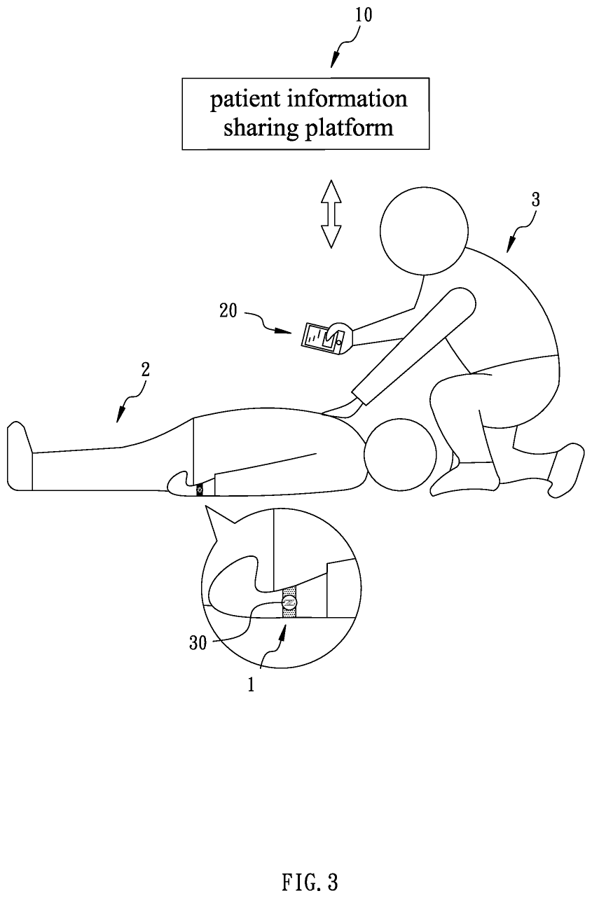

Referring to FIG. 2 and FIG. 3, when an accident occurs, the user 3 may be a medical personnel or a rescue commander. The medical personnel wears the indication device 1 on the patient 2, confirms the identity of the patient 2, and carries out a medical examination upon the patient 2. Next, the medical personnel establishes the identity and injury status of the patient 2 in the patient data 11 and patient status data 12 through the terminal device 20.

The rescue commander is then allowed to use the terminal device 20 to acquire the identity and the injury status of each patient 2 at the scene of accident. Also, based on the current location signal transmitted from the positioning module 22 of the terminal device 20 to the location module 14, the rescue commander is able to know the location of each medical personnel and patient 2, so as to determine the rescue needed locations.

Further, according to each treatment end information 151 and the amount and injury levels of the patients 2, the rescue commander is able to arrange each patient 2 to be delivered to the suitable hospital for receiving treatment, and also establish the treatment end information 151 arranged for the patient 2 in the patient status data 12. The medical staff in the hospital are allowed to use the terminal device 20 for acquiring the patient data 11 and patient status data 12 of the patients 2 from the patient status data 12 in the patient information sharing platform 10 in advance. Also, the family member of a respective patient 2 is able to acquire the treatment end information 151 arranged for the patient 2 from the patient status data 12 in the patient information sharing platform 10 through the terminal device 20.

When the patient 2 is delivered to the hospital, the medical staff in the hospital are able to efficiently identify and confirm the treatment status of the patient 2 through the terminal device 20, thus providing a suitable treatment process, facilitating a highly efficient treatment.

Although particular embodiments of the invention have been described in detail for purposes of illustration, various modifications and enhancements may be made without departing from the spirit and scope of the invention. Accordingly, the invention is not to be limited except as by the appended claims.

What is claimed is:

1. A patient information system comprising:
a patient information sharing platform set up on a server for storing a patient data and a patient status data;
a terminal device connected with the patient information sharing platform through the internet, the terminal device comprising a communication sensing module; and
an identification label providing a sensing identification function and an data transaction function, wherein when the identification label is sensed to be connected with the communication sensing module, the terminal device is able to acquire and edit the patient data and the patient status data.

2. The patient information system of claim 1, wherein the identification label is disposed on an indication device worn on a patient, the communication sensing module connected with the identification label through a near field communication connection.

3. The patient information system of claim 2, wherein the patient data includes the identification label assigned to the patient.

4. The patient information system of claim 1, wherein the patient information sharing platform comprises a patient data module, the patient data module stores the patient data and the patient status data.

5. The patient information system of claim 1, wherein the patient data comprises an identification data and a treatment record data.

6. The patient information system of claim 1, wherein the patient information sharing platform comprises a location module, and the terminal device comprises a positioning module, such that the positioning module acquires a current location signal of the terminal device, and the terminal device transmits the current location signal to the location module.

7. The patient information system of claim 6, wherein the location module transmits the current location signal to the patient status data.

8. The patient information system of claim 1, wherein the patient information sharing platform comprises a hospital data module, the hospital data module comprises a plurality of treatment end information, the patient status data has one of the corresponding treatment end information.

9. The patient information system of claim 1, wherein the patient information sharing platform comprises an account module, the terminal device provides access for an user to operate the patient information sharing platform through the internet, and the user registers a user information with the account module.

10. The patient information system of claim 9, wherein the patient information sharing platform comprises an authorization module, the authorization module limits an authority of the user for viewing data on the patient information sharing platform according to the user information in the account module.
